(19) United States
(12) Patent Application Publication
(10) Pub. No.: US 2026/0166533 A1
(43) Pub. Date: Jun. 18, 2026

(54) MOR FRAMEWORK-TYPE MOLECULAR SIEVES, AND METHODS OF MAKING AND USING THE SAME

(71) Applicant: BRASKEM AMERICA, INC., Philadelphia, PA (US)

(72) Inventors: Ishant Khurana, Pittsburgh, PA (US); Dan Xie, Pittsburgh, PA (US); Graham Gregorich, Pittsburgh, PA (US); Richard Opfer, Pittsburgh, PA (US)

(21) Appl. No.: 19/417,689

(22) Filed: Dec. 12, 2025

Related U.S. Application Data
(60) Provisional application No. 63/733,923, filed on Dec. 13, 2024.

(51) Int. Cl. B01J 29/18; B01J 37/08; C07C 5/333

(52) U.S. Cl. CPC B01J 29/185; B01J 37/08; C07C 5/333; C07C 2529/18

(57) ABSTRACT

The present disclosure provides an MOR framework-type molecular sieve comprising a plurality of crystallites of a mordenite (MOR) framework-type zeolite and a structure-directing composition (SDC) present within the zeolite's pores. The crystallites may have an average crystal size of 50 nm or below. The structure-directing composition may include 2-pyrrolidone or its N-alkyl derivative and at least two different quaternary ammonium cations.

### CROSS-REFERENCE TO RELATED APPLICATIONS

This application claims benefit of priority under 35 U.S.C. § 119(e) to U.S. Provisional Patent Application No. 63/733,923, filed on Dec. 13, 2024, which is hereby incorporated in by reference its entirety.

### FIELD OF INVENTION

The present disclosure relates to zeolite molecular sieves, and more particularly to novel MOR framework-type zeolite molecular sieves, methods for their synthesis, and their use in catalytic applications.

### BACKGROUND

Zeolites are crystalline aluminosilicate materials with well-defined pore structures that have found widespread use in various industrial applications, including catalysis, adsorption, and ion exchange. Among the different zeolite framework types, mordenite (MOR) has attracted significant attention due to its unique one-dimensional pore system and high thermal stability.

MOR framework-type zeolites typically consist of 12-membered ring channels running parallel to the c-axis, interconnected by 8-membered ring side pockets. This pore structure gives MOR zeolites distinctive shape-selective properties, making them valuable in catalytic processes such as hydrocarbon isomerization, alkylation, and cracking reactions.

Conventional synthesis methods for MOR zeolites often involve the use of inorganic structure-directing agents, such as sodium or potassium ions, in combination with high-temperature hydrothermal conditions. However, these traditional approaches can lead to limitations in controlling crystal size, morphology, and silicon-to-aluminum ratio, which are factors affecting the catalytic performance and stability of the resulting materials.

Recent advancements in zeolite synthesis have explored the use of organic structure-directing agents (OSDAs) to tailor the properties of MOR zeolites. OSDAs can influence the nucleation and growth processes, potentially leading to materials with improved characteristics such as smaller crystal sizes, higher crystallinity, and enhanced catalytic activity.

The development of novel synthesis strategies for MOR framework-type zeolites remains an active area of research, with ongoing efforts to optimize their physicochemical properties for specific applications. Particularly, there is interest in methods that can produce MOR zeolites with controlled crystal size, high silicon-to-aluminum ratios, and improved accessibility to active sites.

Zeolite-based catalysts play a crucial role in many industrial processes, including the production of light olefins from alkane feedstocks. The ability to fine-tune the properties of MOR zeolites could potentially lead to more efficient and selective catalysts for such reactions, addressing the growing demand for sustainable chemical production methods.

### SUMMARY

According to an aspect of the disclosure, an MOR framework-type molecular sieve is provided. The molecular sieve includes a plurality of crystallites of a mordenite (MOR) framework-type zeolite, wherein the crystallites have an average crystal size of 50 nm or below, as measured by X-ray powder diffraction. The molecular sieve also includes a structure-directing composition (SDC) present within the zeolite's pore structures, wherein the structure-directing composition comprises (1) 2-pyrrolidone or its N-alkyl derivative, and (2) at least two different quaternary ammonium cations, each being represented by N⁺R₁R₂R₃R₄, wherein R₁, R₂, R₃, and R₄ are each independently an alkyl or aryl.

According to another aspect of the disclosure, an MOR framework-type molecular sieve is provided. The molecular sieve includes a plurality of crystallites of a mordenite (MOR) framework-type zeolite, wherein the crystallites exhibit an X-ray powder diffraction having at least three peaks at the following 2θ diffraction angles: 6.4, 8.5, 9.6, 13.4, 13.8, 14.5, 15.1, and 19.5°2θ±0.2°2θ. The molecular sieve also includes a structure-directing composition (SDC) present within the zeolite's pore structures, wherein the structure-directing composition comprises (1) 2-pyrrolidone or its N-alkyl derivative, and (2) at least two different quaternary ammonium cations, each being represented by N⁺R₁R₂R₃R₄, wherein R₁, R₂, R₃, and R₄ are each independently an alkyl or aryl.

The MOR framework-type molecular sieve may include one or more of the following features. In component (2) of the structure-directing composition, R₁, R₂, R₃, and R₄ may each be independently C₁-C₁₆ alkyl or benzyl, and R₁, R₂, R₃, and R₄ may be the same, or at least one of R₁, R₂, R₃, and R₄ may be different than the others. The structure-directing composition may comprise (1) 2-pyrrolidone and (2) a first quaternary ammonium cation wherein R₁, R₂, R₃, and R₄ are the same, and a second quaternary ammonium cation wherein at least one of R₁, R₂, R₃, and R₄ is different than the others. Component (2) of the structure-directing composition may comprise a tetraethylammonium (TEA) cation and a cetyltrimethylammonium (CTA) cation. The zeolite may comprise silicon oxide (SiO₂) and aluminum (Al₂O₃), and the SiO₂:Al₂O₃ molar ratio in the zeolite may be about 20 to about 50.

According to another aspect of the disclosure, a method for preparing a molecular sieve having a MOR framework type is provided. The method includes providing a mixture comprising: a zeolite of a FAU framework-type, a source of hydroxide ions (OH⁻), a source of alkali or alkaline earth metal ions (M), a structure-directing composition (SDC) comprising (1) 2-pyrrolidone or its N-alkyl derivative, and (2) at least two different quaternary ammonium cations, each being represented by N⁺R₁R₂R₃R₄, wherein R₁, R₂, R₃, and R₄ are each independently an alkyl or aryl, and water. The method also includes subjecting the mixture to crystallization conditions sufficient to form a plurality of crystallites of an MOR framework-type zeolite having the structure-directing composition within the zeolite's pore structures.

The method may include one or more of the following features. The method may further comprise a seed crystal of molecular sieve having a MOR framework type. In component (2) of the structure-directing composition, R₁, R₂, R₃, and R₄ may each be independently C₁-C₁₆ alkyl or benzyl, and R₁, R₂, R₃, and R₄ may be the same, or at least one of R₁, R₂, R₃, and R₄ may be different than the others. The structure-directing composition may comprise (1) 2-pyrrolidone and (2) a first quaternary ammonium cation wherein R₁, R₂, R₃, and R₄ are the same, and a second quaternary ammonium cation wherein at least one of R₁, R₂, R₃, and R₄ is different than the others. Component (2) of the structure-directing composition may comprise a tetraethylammonium (TEA) cation and a cetyltrimethylammonium (CTA) cation. The components of the mixture may be characterized by specific molar ratios. The crystallization conditions may comprise heating the mixture at a temperature ranging from about 120° C. to 180° C. The method may further comprise calcining the formed crystallites of a MOR framework-type zeolite at a temperature of about 400° C. or above for a time sufficient to remove at least part of the structure-directing composition. The method may further comprise replacing the metal ions of the formed crystallites of a MOR framework-type zeolite with one or more cations selected from the group consisting of hydrogen, rare earth metals, and Group 2 to 15 metals.

According to another aspect of the disclosure, a process for producing one or more olefins is provided. The process includes reacting an alkane feedstock in the presence of the MOR framework-type molecular sieve to convert the alkane to one or more olefins, via oxidative dehydrogenation reaction.

The process may include one or more of the following features. The alkane feedstock may be a C₂-C₄ alkane, and the olefin may be a C₂-C₄ olefin. The alkane feedstock may be ethane, and the olefin may be ethylene, or the alkane feedstock may be propane, and the olefin may be propylene, or the alkane feedstock may be butane, and the olefin may be butylene. The reaction may be carried out at a temperature of about 400° C. or below. The reaction may have an olefin formation rate improved by at least 50% compared to a reaction carried out under the same conditions, but using a conventional MOR framework-type molecular sieve with the same metal loading.

### BRIEF DESCRIPTION OF FIGURES

FIG. 1 illustrates a powder X-ray diffraction pattern of the as-synthesized product of Example 1.

FIG. 2 depicts a graph comparing olefin formation rates of the claimed catalyst versus the commercial microporous catalyst over time.

### DETAILED DESCRIPTION

The present disclosure relates to MOR framework-type molecular sieves and their applications in various chemical processes. MOR framework-type molecular sieves, also known as mordenite zeolites, are crystalline aluminosilicate materials with a porous structure. These molecular sieves may have significant industrial applications due to their catalytic properties, high thermal stability, and shape selectivity. For instance, MOR framework-type molecular sieves may be used as catalysts for a variety of catalytic applications, including the conversion of alkanes to olefins, and petrochemical productions using novel routes to reduce carbon footprint and renewable chemical conversions. The enhanced performance of these MOR framework-type molecular sieves may be attributed to their structural characteristics and composition, which will be described in more detail in the following sections.

The MOR framework-type molecular sieves disclosed herein may have a particular structure and composition that contributes to their enhanced catalytic performance. These molecular sieves may comprise a plurality of crystallites of a mordenite (MOR) framework-type zeolite. In some cases, the crystallites may have an average crystal size of 50 nm or below (such as 45 nm or below, 40 nm or below, 35 nm or below, 30 nm or below, or 25 nm or below), as measured by X-ray powder diffraction.

The MOR framework-type molecular sieves may include a structure-directing composition (SDC) present within the zeolite's pore structures. This SDC may comprise two main components: (1) 2-pyrrolidone or its N-alkyl derivative, such as N-methyl-2-pyrrolidone, and (2) at least two different quaternary ammonium cations. In some cases, it has been found that such a combination of organic species produces nanocrystal forms of the product. For instance, smaller crystallites may provide a larger external surface area and shorter diffusion paths for reactants and products, potentially enhancing the overall reaction rate.

The quaternary ammonium cations in the SDC may be represented by the general formula N⁺R₁R₂R₃R₄, where R₁, R₂, R₃, and R₄ are each independently an alkyl or aryl group. In some cases, R₁, R₂, R₃, and R₄ may be C₁-C₁₆ alkyl (such as C₁-C₈ alkyl, C₁-C₆ alky, C₁-C₄ alkyl, or C₂-C₄ alkyl) or benzyl groups. The R groups may be the same, or at least one of R₁, R₂, R₃, and R₄ may be different than the others.

In some cases, the SDC may comprise 2-pyrrolidone, a first quaternary ammonium cation where R₁, R₂, R₃, and R₄ are the same, and a second quaternary ammonium cation where at least one of R₁, R₂, R₃, and R₄ is different than the others.

For example, the SDC may include a tetraethylammonium (TEA) cation (such as a salt of TEA, e.g., tetraethylammonium bromide (TEABr), tetraethylammonium chloride (TEACl), tetraethylammonium hydroxide (TEAOH), or a mixture thereof) and a cetyltrimethylammonium (CTA) cation (such as a salt of CTA, e.g., cetyltrimethylammonium bromide (CTAB), cetyltrimethylammonium chloride (CTAC), cetyltrimethylammonium hydroxide (CTAOH), or a mixture thereof).

One of the advantages of the disclosed MOR framework-type molecular sieves is that the organic templates have larger molecular sizes than the inorganic templates. Therefore, to fill in the same voids (i.e., pore configuration) inside the same porous material, less amount of the organic cations/species (compared to inorganic species) is needed to serve as pore filling agents. This, in turn, allows the production of MOR with less aluminum inside the framework structure of the MOR material to counter-balance the charge of the framework needed.

Note that Al in the zeolite framework is 3+ charge while Si is Si4+ charge. Replacing one Si with one Al will introduce one negative charge into the zeolite framework. Therefore, to balance the negative charge by external cations, inorganic cations, such as Na+, or organic cations, such as tetraethylammonium (which is 1+) may be used. The latter organic cation, however, is much larger in size compared to inorganic cations like Na+ or K+, which leads to higher SAR (silica-to-alumina ratio) of the product. The SAR is an important parameter of zeolites, among others, in determining the acidic property of the material.

In one embodiment, the zeolite component of the MOR framework-type molecular sieve comprises silicon oxide (SiO₂) and aluminum oxide (Al₂O₃). The SiO₂:Al₂O₃ molar ratio in the zeolite may range from about 20 to about 50 (such as about 20 to about 40, about 20 to about 30, or about 20 to about 25). In some cases, the MOR framework-type molecular sieve may have a silica-to-alumina ratio (SAR) of about 24.

FAU zeolite may be used as both the Si source and Al source for making another type of zeolites—in this case, MOR. This approach is called “interzeolite conversion.” While not all zeolites can be made in such a route, the benefit of such a route include (1) faster synthesis time; (2) different Al sitting inside the zeolite framework (and therefore different acid strengths and property) when compared to the traditional approach where an Si source and Al source are coming from independent reagents (i.e., one or more reagents solely provide Si source, and one or more reagents solely provide Al source); and generally (3) smaller crystal sizes.

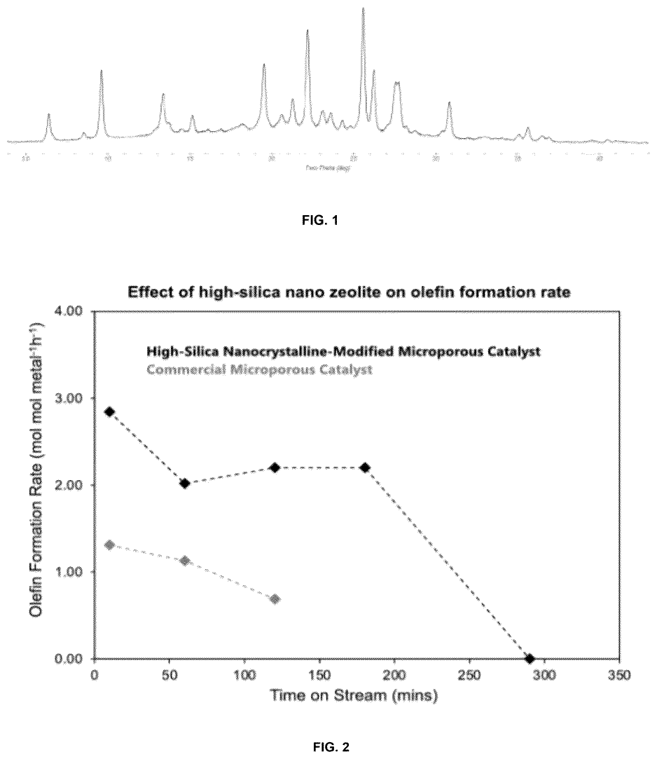

The crystallites of the MOR framework-type zeolite may exhibit a characteristic X-ray powder diffraction pattern. FIG. 1 shows an example of such a pattern. In one embodiment, the X-ray powder diffraction pattern of the disclosed MOR framework-type molecular sieve exhibits at least three peaks (such as at least four peaks, at least five peaks, or at least six peaks) at specific 2θ diffraction angles. As depicted in FIG. 1, these angles may include 6.4, 8.5, 9.6, 13.4, 13.8, 14.5, 15.1, and 19.5°2θ±0.2°2θ.

The X-ray powder diffraction pattern can also be used to determine the crystal size of the MOR framework-type molecular sieves. The Scherrer Equation is used to relate the peak broadening (measured as full width at half-maximum, FWHM) to the average crystallite size.

Broader peaks indicates smaller crystals and vice versa. The Scherrer Equation is shown below:

D=(K*λ)/(β*cos θ) where: D=average crystallite size; K=shape factor; λ=X-ray wavelength; β=FWHM of the diffraction peak in radians; θ=Bragg diffraction angle.

By way of example, using the Rigaku powder X-ray diffractometer to collect the XRD data, and a copper anode to generate X-rays, the X-ray wavelength λ, is 1.54 angstrom, i.e., 1.54*10\hat{ }(−10) m; using the [200]-peak that is located at 9.62 degree of 2θ position for the calculation, so here θ is 9.62/2=4.81 degree, which is 4.81*pi/180=0.084 radians; the FWHM value, β, is 0.22 degree, which is 0.22*pi/180=0.00384 radians; and the K shape factor is 0.89.

Putting these numbers in the above-noted equation:

$$\begin{matrix}D = ( K \times λ ) / ( β \times cos θ ) \\ D = ( 0.89 \times 1.54 \times 10 \hat{} ( - 10 ) m ) / ( 0.00384 \times cos ( 0.084 ) ) \\ D = ( 1.371 \times 10 \hat{} ( - 10 ) m ) / ( 0.00384 \times 0.9969 ) \\ D \approx ( 1.371 \times 10 \hat{} ( - 10 ) m ) / 0.00383 ) \\ D \approx 3.58 \times 10 \hat{} ( - 8 ) m = 35.8 nm\end{matrix}$$

The calculated average crystalline size is thus 35.8 nm.

The MOR framework-type molecular sieves disclosed herein may exhibit physical properties that contribute to their performance. For example, in some cases, the MOR framework-type molecular sieve may have a surface area of greater than about 350 cm⁻²/g (e.g., greater than about 360 cm⁻²/g or greater than about 370 cm⁻²/g), as measured by Ar adsorption isotherms obtained at 87K on a Surface Area and Porosity Analyzer. Additionally, the MOR framework-type molecular sieve may have a micropore volume of about 0.15 to 0.30 cm⁻³/g (e.g., from about 0.15 to 0.25 cm⁻³/g, or ranging from about 0.18 to 0.22 cm⁻³/g), measured using the same method.

The MOR framework-type molecular sieves described herein may be prepared according to methods involving the use of precursors, reaction conditions, and post-synthesis treatments, as described in more detail in subsequent sections of this disclosure.

In some cases, the synthesis of MOR framework-type molecular sieves may begin with providing a mixture comprising several components. The mixture may include a zeolite of a FAU framework-type, such as Zeolite Y. In some cases, CBV720 powders may be used as the FAU framework-type zeolite source. The mixture may also include a source of hydroxide ions (OH—), which may be provided by sodium hydroxide solution. A source of alkali or alkaline earth metal ions (M) may also be included in the mixture.

The mixture may further comprise a structure-directing composition (SDC). The SDC may include 2-pyrrolidone or its N-alkyl derivative, such as N-methyl-2-pyrrolidone. Additionally, the SDC may include at least two different quaternary ammonium cations, as described above. In some cases, the SDC may comprise tetraethylammonium (TEA) cations and cetyltrimethylammonium (CTA) cations, as described above. These components may be provided in the form of tetraethylammonium hydroxide solution and cetyltrimethylammonium bromide solid, respectively.

Water may also be included in the mixture. In some cases, deionized water may be used.

The synthesis process may involve subjecting the mixture to crystallization conditions sufficient to form a plurality of crystallites of an MOR framework-type zeolite having the structure-directing composition within the zeolite's pore structures. The crystallization conditions may comprise heating the mixture at a temperature ranging from about 120° C. to 180° C. (such as from about 140 to about 160° C., or from about 150 to about 160° C.). In some cases, the mixture may be heated in an autoclave reactor.

After the crystallization process, the resulting solid product may be separated from the solution through filtration. The solid product may then be washed with deionized water and dried at a temperature of about 95° C.

In some cases, a seed crystal of molecular sieve having a MOR framework type may be added to the mixture. The amount of seed crystal may range from 0.01 to 10,000 ppm (e.g. from 100 to 5000 ppm, or from 250 to 2500) by weight of the mixture.

The components of the mixture may be characterized by specific molar ratios. In some cases, one or more of these molar ratios, or combinations thereof, may be:
SiO₂:Al₂O₃: ≥20 (e.g., 20 to 50, 20 to 40, or 25 to 35),
SDC:SiO₂: 0.05 to 0.60 (e.g., 0.05 to 0.40, 0.05 to 0.30, 0.10 to 0.30),
M:SiO₂: 0.05 to 0.50 (e.g., 0.05 to 0.30, 0.05 to 0.20, 0.15 to 0.50, 0.15 to 0.30, or 0.15 to 0.20),
OH⁻:SiO₂: 0.10 to 0.50 (e.g., 0.15 to 0.50, or 0.20 to 0.45), and
H₂O:SiO₂: 3 to 60 (e.g., 5 to 60, 5 to 40, 5 to 20, 5 to 10, 10 to 60, 10 to 40, 10 to 20, 15 to 60, or 15 to 40).

The formed crystallites of the MOR framework-type zeolite may then undergo a calcination process. This process may involve heating the crystallites at a temperature of about 400° C. or above (such as about 450° C. or above, about 500° C. or above, about 550° C. or above, or about 550° C.) for a time sufficient to remove at least part of the structure-directing composition. In some cases, the calcination may involve heating to 550° C. at a rate of 1.5° C./minute and holding at 550° C. for 5 hours.

Following calcination, the MOR framework-type molecular sieve may undergo an ion exchange process. This process may involve replacing the metal ions in the formed crystallites with one or more cations.

In some cases, the crystallites may be subjected to hydrogen ions or a hydrogen precursor, such as ammonium ions (e.g., an ammonium nitrate solution), to replace the metal ions in the zeolite with hydrogen ions. By way of example, this ion exchange treatment may use a 0.1 N ammonium nitrate solution at 95° C. for 2 hours, and may be repeated two or three times.

In other cases, the MOR framework-type molecular sieve may be loaded with rare earth metals or Group 2 to 15 metals (e.g., transition metals such as Group 11 metal, e.g., Cu) to load the rare earth metals or Group 2 to 15 metals to the zeolite. By way of example, copper may be incorporated via a solid-state ion-exchange process in a process that involves physically mixing a copper nitrate precursor with the proton form of the MOR molecular sieve.

After the ion exchange process, a final calcination step may be performed to convert the zeolite to its proton form. This may involve heating to 400° C. at a rate of 2° C./min and holding for 3 hours.

In one embodiment, this invention relates to a method for preparing a molecular sieve having a MOR framework type, as noted above, in which the structure-directing composition comprises (1) 2-pyrrolidone and (2) a tetraethylammonium (TEA) cation (such as a salt of TEA, e.g., tetraethylammonium bromide (TEABr), tetraethylammonium chloride (TEACl), tetraethylammonium hydroxide (TEAOH), or a mixture thereof), and a cetyltrimethylammonium (CTA) cation (such as a salt of CTA, e.g., cetyltrimethylammonium bromide (CTAB), cetyltrimethylammonium chloride (CTAC), cetyltrimethylammonium hydroxide (CTAOH), or a mixture thereof); the crystallization conditions comprise heating the mixture at a temperature ranging from about 120° C. to 180° C. (such as from about 140 to about 160° C., or from about 150 to about 160° C.), optionally in an autoclave reactor; and the method further comprises: calcining the formed crystallites of a MOR framework-type zeolite at a temperature of about 400° C. or above (such as about 450° C. or above, about 500° C. or above, about 550° C. or above, or about 550° C.) for a time sufficient to remove at least part of the structure-directing composition; subjecting the formed crystallites of a MOR framework-type zeolite to hydrogen ions or a hydrogen precursor (such as ammonium ions, e.g., ammonium nitrate solution) to replace the metal ions in the zeolite with hydrogen ions; and subjecting the formed crystallites of a MOR framework-type zeolite to rare earth metals or Group 2 to 15 metals (e.g., transition metals such as Group 11 metal, e.g., Cu), or their precursors, to load the rare earth metals or Group 2 to 15 metals (e.g., transition metals such as Group 11 metal, e.g., Cu) to the zeolite.

In another embodiment, the invention relates to MOR framework-type molecular sieves prepared according to any of the above-disclosed methods.

As noted above, the enhanced performance of these MOR framework-type molecular sieves may be attributed to their structural characteristics and composition. These improvements may lead to more efficient and economical olefin production processes, potentially offering significant advantages in industrial applications. In particular, the application of the MOR framework-type molecular sieves is useful for light olefin production via low-temperature oxidative dehydrogenation of low-carbon alkanes. The catalytic performance of the particular MOR framework-type molecular sieves for this application outperforms the commercial MOR catalyst.

In some embodiments of the invention, the MOR framework-type molecular sieves may be used in processes for producing olefins from alkane feedstocks. The alkane feedstock may be a C₂-C₄ alkane, and the resulting olefin may be a corresponding C₂-C₄ olefin. For example, the alkane feedstock may be ethane, and the olefin produced may be ethylene. In other cases, the alkane feedstock may be propane, and the olefin produced may be propylene. Additionally, the alkane feedstock may be butane, and the olefin produced may be butylene.

As an example, when the alkane feedstock is ethane, and the olefin product is the ethane feed concentration may be about 35% in nitrogen, and the total feed rate for the ethane dehydrogenation reaction may be about 200 ml/min. These conditions may be varied according to the feedstock, and various other factors appreciated by one skilled in the art.

The process for producing one or more olefins may involve reacting an alkane feedstock in the presence of the MOR framework-type molecular sieve to convert the alkane to one or more olefins via oxidative dehydrogenation reaction. This reaction may be carried out at a temperature of about 400° C. or below. In some cases, the reaction temperature may be even lower, such as about 350° C. or below.

The MOR framework-type molecular sieves disclosed herein may exhibit improved catalytic performance compared to conventional MOR framework-type molecular sieves. In some cases, the reaction using the disclosed MOR framework-type molecular sieves may have an olefin formation rate improved by at least 50% (such as at least 60%, at least 70%, at least 80%, at least 90%, or at least 100%), compared to a reaction carried out under the same conditions but using a conventional MOR framework-type molecular sieve with the same metal loading. This improvement in olefin formation rate may be even higher in some instances, potentially reaching improvements of at least 1.5 fold (such as at least 2 fold, at least 2.5 fold, at least 3 fold, at least 3.5 fold, at least 4 fold, or at least 5 fold), compared to a reaction carried out under the same conditions, but using a conventional MOR framework-type molecular sieve with the same metal loading.

The synthesis method described herein may result in MOR framework-type molecular sieves with specific physical properties. For characterization purposes, the catalyst samples may be pelleted and sieved to a nominal diameter between 180-250 μm. The surface area and porosity of the samples may be analyzed using specific degassing processes, which may involve heating under vacuum conditions.

The synthesis method and post-synthesis treatments described herein may contribute to the unique properties and enhanced performance of the resulting MOR framework-type molecular sieves. These molecular sieves may exhibit improved catalytic activity in various chemical processes, including the production of olefins from alkane feedstocks.

The MOR framework-type molecular sieves described herein may be used in various catalytic applications, particularly in the production of olefins from alkane feedstocks. In some cases, these molecular sieves may exhibit superior catalytic performance compared to conventional MOR framework-type molecular sieves.

The specific properties of the MOR framework-type molecular sieve, such as its pore structure, surface area, and acidity, may contribute to its effectiveness in these catalytic applications. The molecular sieve may provide active sites for the oxidative dehydrogenation reactions while minimizing undesired side reactions.

In some implementations, the reaction conditions, such as temperature, feed concentration, and flow rate, may be optimized for each specific alkane feedstock to maximize olefin yield and selectivity. The ability to operate at lower temperatures, such as 350° C. or below, may offer advantages in terms of energy efficiency and catalyst longevity, as noted above.

The MOR framework-type molecular sieve may interact with alkane feedstocks to catalyze the production of olefins through oxidative dehydrogenation reactions. The structure and composition of the molecular sieve may contribute to its catalytic performance in these reactions.

In some cases, the crystalline structure of the MOR framework-type zeolite may provide a arrangement of pores and channels that allow for the selective adsorption and reaction of alkane molecules. The pore structure may influence the diffusion of reactants and products, potentially affecting the overall reaction kinetics and product selectivity.

The structure-directing composition, comprising 2-pyrrolidone or its N-alkyl derivative and a quaternary ammonium cation, may play a role in shaping the molecular sieve's structure during synthesis. This composition may influence the formation of specific pore sizes and arrangements, which may in turn affect the catalytic properties of the resulting molecular sieve.

In some cases, the presence of the structure-directing composition within the zeolite's pore structures may create specific active sites for the oxidative dehydrogenation reactions.

These active sites may facilitate the breaking of C—H bonds in alkane molecules and the formation of C═C bonds to produce olefins.

The SiO₂:Al₂O₃ molar ratio in the zeolite may affect its acidity and hydrophobicity. A higher ratio may result in fewer acid sites and a more hydrophobic surface, which may influence the adsorption and reaction of alkane molecules on the catalyst surface.

In some cases, the surface area and micropore volume of the MOR framework-type molecular sieve may contribute to its catalytic performance. A larger surface area may provide more active sites for the reaction, while the micropore volume may affect the accessibility of these sites to reactant molecules.

The crystallite size of the MOR framework-type zeolite may also play a role in its catalytic activity. Smaller crystallites may provide a larger external surface area and shorter diffusion paths for reactants and products, potentially enhancing the overall reaction rate.

In some cases, the metal ions present in the molecular sieve, whether from the original synthesis or introduced through post-synthesis treatments, may serve as active sites for the oxidative dehydrogenation reactions. These metal ions may facilitate the activation of oxygen and the abstraction of hydrogen from alkane molecules.

The combination of these structural and compositional features may result in a molecular sieve with high selectivity and activity for alkane-to-olefin conversions. The specific arrangement of pores, active sites, and metal ions may allow for efficient conversion of alkanes to olefins while minimizing undesired side reactions.

In some cases, the stability of the MOR framework-type molecular sieve under reaction conditions may contribute to its sustained catalytic performance over time. The robust structure may resist degradation at elevated temperatures and in the presence of reactant and product molecules.

The interaction between the alkane feedstock and the molecular sieve may involve several steps. The alkane molecules may first adsorb onto the surface or within the pores of the molecular sieve. The adsorbed alkanes may then interact with active sites, potentially including metal ions or acid sites, which may facilitate the breaking of C—H bonds. Simultaneously, oxygen may be activated on the catalyst surface. The activated oxygen species may then abstract hydrogen from the alkane molecules, leading to the formation of olefin products.

In some cases, the specific pore structure of the MOR framework-type molecular sieve may influence the product distribution by favoring the formation of certain olefins based on shape selectivity. The pore dimensions may allow for the preferential diffusion of specific product molecules, potentially enhancing the selectivity towards desired olefins.

The overall catalytic performance of the MOR framework-type molecular sieve in alkane-to-olefin conversions may result from the interplay of these various structural, compositional, and mechanistic factors. The specific combination of features in the molecular sieve may provide a catalytic environment that enables efficient and selective olefin production under relatively mild conditions.

To evaluate the catalytic performance of the MOR framework-type molecular sieves, experiments may be conducted using a lab-scale plug flow reactor. In some cases, 0.2 g of catalyst is used in the reactor, although this amount may be varied according to various conditions and preferences, as one skilled in the art would appreciate.

### EXAMPLES

### Example 1: Synthesis of MOR Type Framework Molecular Sieve

4.45 grams of deionized water, 0.47 grams of 50 wt % sodium hydroxide solution, 2.76 grams of 25 wt % Tetraethylammonium hydroxide solution (Sigma-Aldrich), and 1.50 grams of CBV720 powders (Zeolyst Y zeolite) were mixed and stirred for 2 hours. Then, 1.00 grams of 2-Pyrrolidinone (Sigma-Aldrich) was added dropwise into the above gel under vigorous stirring for 2 hours. Finally, 0.43 grams of Cetyltrimethylammonium bromide solid (CTAB, Sigma-Aldrich) was added to the gel and stirred for at least 4 hours to create a homogeneous solution. The solution was transferred to a Teflon liner and sealed into a 23 mL autoclave from Parr Instrument company. A hydrothermal treatment was then carried out in an oven at 150° C. for 4 days, with the autoclave rotating at ˜50 rpm. The resulting solid product was separated from the solution through filtration, followed by washing with deionized water and dried at 95° C.

FIG. 1 shows the powder X-ray diffraction (XRD) patterns of the as-synthesized aluminosilicate product of Example 1. As depicted in FIG. 1, the powder XRD pattern of the as-synthesized product showed the typical phase of MOR topology and also indicated decreased crystal size as inferred from the peak broadening in the XRD pattern.

### Example 2: Converting the Products from as-Synthesized Form to Proton Form

The as-synthesized molecular sieve product of Example 1 was calcined inside a muffle furnace under a flow of air heated to 550° C. at a rate of 1.5° C./minute and held at 550° C. for 5 hours, cooled and then analyzed by powder XRD. The powder XRD data indicated that the material remains stable after calcination to remove the organic species.

After calcination, the products were treated with 0.1N ammonium nitrate solution at 95° C. for 2 hours. The solution was cooled, decanted, dried at 95 C and this process was repeated three times. The powders were then calcined again in a muffle furnace in the presence of air, heated to 400° C. at a rate of 2° C./min and held for 3 hours to convert the zeolite sample from ammonium form to proton form.

### Example 3: Metal Loading to Molecular Sieve

For the incumbent reference molecular sieve catalyst, Cu was incorporated on commercial MOR type framework molecular sieve (CBV21A from Zeolyst) via solid-state ion-exchange, wherein 0.08 g of copper nitrate precursor was physically mixed with 5.92 g of proton form of MOR molecular sieve and grounded in a mortar and pestle for about 15 mins or till the solid mixture turns light green or seems homogenous. This solid mixture was then heated to 873K (0.167 K s-1) under the flow of air in a horizontal tube furnace and held for 6 hours before cooling to room temperature.

For the MOR type molecular sieve obtained from Example 2, Cu was supported on the novel product using the same solid-state ion-exchange procedure.

### Example 4: Catalyst Characterization

Surface area and micropore volumes of the catalysts were determined from Ar adsorption isotherms measured at 87 K on a Micromeritics ASAP 2020 Surface Area and Porosity

Analyzer. Typically, 0.03-0.05 g of pelleted and sieved sample (nominal diameter between 180-250 m) were degassed by heating to 1200 C (100 C/min) under vacuum (<5 μmHg) for 2 h, and then further heating to 3500 C (100 C/min) under vacuum (<5 μmHg) and holding for 9 h. Volumetric gas adsorption within micropores (cm3 g-1 at STP) was estimated from analysis of semi-log derivative plots of the adsorption isotherm (∂(Vads)/∂(ln(P/P0)) vs. ln(P/P0)) to identify the micropore filling transition (first maximum) and then the end of micropore filling (subsequent minimum). Micropore volumes (cm3 g-1) were obtained by converting standard gas adsorption volumes (cm3 gcat-1 at STP) to liquid volumes using a density conversion factor assuming the liquid density of Ar at −1860 C. Surface area and micropore volumes of the catalysts are shown in Table 1 and are in line with what is expected for conventional zeolite molecular sieves. The results on the silica-to-alumina ratio (SAR) of the samples were collected by ICP elemental analysis.

TABLE 1
Surface area, micropore volume, and silica-to-alumina
ratio (by ICP elemental analysis) measured on conventional
and novel zeolite molecular sieve-based catalysts.

| | | Micropore | SAR (Silica |
|---|---|---|---|
| | SampleSurface | Volume | to Alumina |
| Sample | Area (cm² g⁻¹) | (cm³ g⁻¹) | Ratio) |
| Conventional MOR zeolite | 368 | 0.16 | 16 |
| (CBV21A from Zeolyst) | | | |
| Novel MOR zeolite | 373 | 0.20 | 24 |
| (Example 2) | | | |

### Example 5: Catalytic Evaluation—Selective Production of Ethylene from Ethane Reaction at Low Temperature

Ethane was fed onto a bed of transition metal incorporated microporous mixed metal oxide (0.2 g) contained in a lab-scale plug flow reactor. The catalyst bed was maintained at temperature of 3500 C. Ethane feed concentration was about 35% in nitrogen and total feed rate was about 200 ml/min.

The novel Cu—H-MOR catalyst (from Example 4) exhibited higher and more sustained activity at 100% selectivity vs. the incumbent Cu—H-MOR catalyst (from Zeolyst). FIG. 2 illustrates the results comparing the ethylene formation rate as a function of time-on-stream at 3500 C on both the incumbent and novel in-house synthesized catalysts.

FIG. 2 illustrates the performance of the MOR framework-type molecular sieves in the production of ethylene from ethane. The graph in FIG. 2 shows the ethylene formation rate as a function of time-on-stream for two different catalysts: a high-silica nanocrystalline-modified microporous catalyst (representing the MOR framework-type molecular sieve described herein) and a commercial microporous catalyst.

As depicted in FIG. 2, the high-silica nanocrystalline-modified microporous catalyst demonstrates a higher ethylene formation rate throughout the measured time period compared to the commercial microporous catalyst. The high-silica catalyst starts with an ethylene formation rate of around 2.8 mmol metal⁻¹ hr⁻¹ and maintains a rate above 2.0 mmol metal⁻¹ hr⁻¹ for about 200 minutes before declining. In contrast, the commercial catalyst shows a lower initial rate of about 1.4 mmol metal⁻¹ hr⁻¹, which steadily decreases to below 1.0 mmol metal⁻¹ hr⁻¹ by 100 minutes.

The performance data illustrated in FIG. 2 suggests that the MOR framework-type molecular sieves described herein may offer significant advantages in olefin production processes. In some cases, the reaction using these MOR framework-type molecular sieves may have an olefin formation rate improved by at least 50% compared to a reaction carried out under the same conditions but using a conventional MOR framework-type molecular sieve with the same metal loading.

The enhanced catalytic performance of these MOR framework-type molecular sieves may be attributed to their unique structural characteristics and composition, as described in previous sections. These improvements may lead to more efficient and economical olefin production processes, potentially offering significant advantages in industrial applications.

1. An MOR framework-type molecular sieve, comprising:
a plurality of crystallites of a mordenite (MOR) framework-type zeolite, wherein the crystallites have an average crystal size of 50 nm or below, as measured by X-ray powder diffraction; and
a structure-directing composition (SDC) present within the zeolite's pore structures, wherein the structure-directing composition comprises (1) 2-pyrrolidone or its N-alkyl derivative, and (2) at least two different quaternary ammonium cations, each being represented by N⁺R₁R₂R₃R₄, wherein R₁, R₂, R₃, and R₄ are each independently an alkyl or aryl.

2. An MOR framework-type molecular sieve, comprising:
a plurality of crystallites of a mordenite (MOR) framework-type zeolite, wherein the crystallites exhibit an X-ray powder diffraction having at least three peaks at the following 2θ diffraction angles: 6.4, 8.5, 9.6, 13.4, 13.8, 14.5, 15.1, and 19.5°2θ±0.2°2θ; and
a structure-directing composition (SDC) present within the zeolite's pore structures, wherein the structure-directing composition comprises (1) 2-pyrrolidone or its N-alkyl derivative and (2) at least two different quaternary ammonium cations, each being represented by N⁺R₁R₂R₃R₄, wherein R₁, R₂, R₃, and R₄ are each independently an alkyl or aryl.

3. The MOR framework-type molecular sieve of claim 1, wherein in component (2) of the structure-directing composition, R₁, R₂, R₃, and R₄ are each independently C₁-C₁₆ alkyl or benzyl; and wherein R₁, R₂, R₃, and R₄ are the same, or at least one of R₁, R₂, R₃, and R₄ is different than the others.

4. The MOR framework-type molecular sieve of claim 3, wherein the structure-directing composition comprises (1) 2-pyrrolidone and (2) a first quaternary ammonium cation wherein R₁, R₂, R₃, and R₄ are the same, and a second quaternary ammonium cation wherein at least one of R₁, R₂, R₃, and R₄ is different than the others.

5. The MOR framework-type molecular sieve of claim 4, wherein component (2) of the structure-directing composition comprises a tetraethylammonium (TEA) cation, and a cetyltrimethylammonium (CTA) cation.

6. The MOR framework-type molecular sieve of claim 1, wherein the zeolite comprises silicon oxide (SiO₂) and aluminum (Al₂O₃), and the SiO₂:Al₂O₃ molar ratio in the zeolite is about 20 to about 50.

7. A method for preparing a molecular sieve having a MOR framework type, comprising:
providing a mixture comprising:
i. a zeolite of a FAU framework-type,
ii. a source of hydroxide ions (OH⁻),
iii. a source of alkali or alkaline earth metal ions (M),
iv. a structure-directing composition (SDC) comprising (1) 2-pyrrolidone or its N-alkyl derivative, and (2) at least two different quaternary ammonium cations, each being represented by N⁺R₁R₂R₃R₄, wherein R₁, R₂, R₃, and R₄ are each independently an alkyl or aryl, and
v. water; and
subjecting the mixture to crystallization conditions sufficient to form a plurality of crystallites of an MOR framework-type zeolite having the structure-directing composition within the zeolite's pore structures.

8. The method of claim 7, further comprising a seed crystal of molecular sieve having a MOR framework type, optionally in an amount of 0.01 to 10,000 ppm by weight of the mixture.

9. The method of claim 7, wherein, in component (2) of the structure-directing composition, R₁, R₂, R₃, and R₄ are each independently C₁-C₁₆ alkyl or benzyl; and wherein R₁, R₂, R₃, and R₄ are the same, or at least one of R₁, R₂, R₃, and R₄ is different than the others.

10. The method of claim 7, wherein the structure-directing composition comprises (1) 2-pyrrolidone and (2) a first quaternary ammonium cation wherein R₁, R₂, R₃, and R₄ are the same, and a second quaternary ammonium cation wherein at least one of R₁, R₂, R₃, and R₄ is different than the others.

11. The method of claim 10, wherein component (2) of the structure-directing composition comprises a tetraethylammonium (TEA) cation, and a cetyltrimethylammonium (CTA) cation.

12. The method of claim 7, wherein the components of the mixture are characterized by the following molar ratios:
SiO₂:Al₂O₃: ≥20,
SDC:SiO₂: 0.05 to 0.60,
M:SiO₂: 0.05 to 0.50,
OH⁻:SiO₂: 0.10 to 0.50, and
H₂O:SiO₂: 3 to 60.

13. The method of claim 7, wherein the crystallization conditions comprise:
heating the mixture at a temperature ranging from about 120° C. to 180° C., optionally in an autoclave reactor.

14. The method of claim 7, further comprising at least one of:
calcining the formed crystallites of a MOR framework-type zeolite at a temperature of about 400° C. or above for a time sufficient to remove at least part of the structure-directing composition; and
replacing the metal ions of the formed crystallites of a MOR framework-type zeolite with one or more cations selected from the group consisting of hydrogen, rare earth metals, and Group 2 to 15 metals.

15-17. (canceled)

18. The method of claim 7, wherein:
the structure-directing composition comprises (1) 2-pyrrolidone and (2) a tetraethylammonium (TEA) cation, and a cetyltrimethylammonium (CTA) cation;
the crystallization conditions comprise heating the mixture at a temperature ranging from about 120° C. to 180° C., optionally in an autoclave reactor; and
the method further comprises:
calcining the formed crystallites of a MOR framework-type zeolite at a temperature of about 400° C. or above for a time sufficient to remove at least part of the structure-directing composition;
subjecting the formed crystallites of a MOR framework-type zeolite to hydrogen ions or a hydrogen precursor to replace the metal ions in the zeolite with hydrogen ions; and
subjecting the formed crystallites of a MOR framework-type zeolite to rare earth metals or Group 2 to 15 metals or their precursors, to load the rare earth metals or Group 2 to 15 metals to the zeolite.

19. A MOR framework-type molecular sieve prepared according to claim 1.

20. The MOR framework-type molecular sieve of claim 1, having a surface area of greater than about 350 cm⁻²/g, measured by Ar adsorption isotherms obtained at 87K on a Surface Area and Porosity Analyzer.

21. The MOR framework-type molecular sieve of claim 1, having a micropore volume ranging from about 0.15 to 0.30 cm⁻³/g measured by Ar adsorption isotherms obtained at 87K on a Surface Area and Porosity Analyzer.

22. A process for producing one or more olefins, comprising:
reacting an alkane feedstock in the presence of the MOR framework-type molecular sieve of claim 1, to convert the alkane to one or more olefins, via oxidative dehydrogenation reaction.

23-26. (canceled)
